(19) United States
(12) Patent Application Publication
(10) Pub. No.: US 2022/0117668 A1
(43) Pub. Date: Apr. 21, 2022

(54) FAST PLANNING SYSTEM AND METHOD APPLICABLE TO ABLATION THERAPY

(71) Applicant: MAGI COMPANY LTD, Shanghai (CN)

(72) Inventors: Fangyu QIN, Shanghai (CN); Kangwei ZHANG, Shanghai (CN); Aili ZHANG, Shanghai (CN); Jincheng ZOU, Shanghai (CN); Jianqi SUN, Shanghai (CN); Xuemin XU, Shanghai (CN)

(21) Appl. No.: 17/422,411

(22) PCT Filed: Jan. 9, 2020
(86) PCT No.: PCT/CN2020/071197

§ 371 Date: Jul. 12, 2021

(30) Foreign Application Priority Data
Jan. 10, 2019 (CN) 201910023929.9

(51) Int. Cl. A61B 34/20; A61B 34/10

(52) U.S. Cl. CPC A61B 34/20; A61B 34/10; A61B 2034/2065; A61B 2034/108; A61B 2034/2063; A61B 2034/107

(57) ABSTRACT

Disclosed are a fast planning system and method applicable to ablation therapy. The system comprises an input device, a calculation device and an output device, the calculation device obtains a boundary of the tumor and an actual position of an ablation probe relative to a tumor through the input device, the quantitative relationship between the ablation range and the ablation parameters of the ablation probe, and maximum ablation range parameters of the ablation probe. The system obtains the ablation parameters of the ablation probe by means of the calculation device based on the obtained information, outputs the ablation parameters by means of the output device, and obtains a revised planning suggestion. The present invention resolves the issue of incomplete ablation in a clinical ablation operation possibly resulting from deviation of an actual probe insertion position from a probe insertion position planned prior to the operation, and is conducive to achieving precision control over thermal dosage at a focal region and ensuring the effectiveness of ablation therapy.

### TECHNICAL FIELD

The application relates to an intraoperative fast planning technique, in particular to an intraoperative fast planning system and method for ablation therapy.

### BACKGROUND

Minimally invasive ablation of tumors has been developed rapidly in recent years. Local ablation of the tumors is a treatment method to accurately locate, inactivate tumor cells in situ, reduce tumor load, relieve local symptoms and improve recovery with the guidance of imaging device. The most commonly used ablation methods mainly comprise cryoablation, radiofrequency ablation and microwave ablation, which have been widely used in the treatment of benign and malignant tumors such as liver, lung, kidney, bone, thyroid, breast and lymph nodes.

Local ablation of the tumors also comprises multi-modal ablation. The multi-modal ablation is a minimally invasive ablation method combining the cryoablation and the radiofrequency ablation. Through rapid changes of temperature and stress in tumor tissues, tumor cells are completely disintegrated, tumor antigens are released to the maximum extent, and irreversible cell damage is caused by the minimally invasive ablation.

However, for the local ablation, accurate and fast planning for thermal dosage is essential if good clinical effects need to be obtained.

On the other hand, the existing technology currently only stays in a preoperative planning stage, that is, before the doctor inserts a probe, providing an appropriate probe insertion path and ablation conditions. However, during the surgical operation of the doctor, due to the patient's breathing displacement, posture change, and the doctor's own operating experience, the actual probe insertion position during the operation deviates from the planned probe insertion position before the operation. At this time, if still performing the ablation according to the originally planned ablation conditions, it may result incomplete ablation. Therefore, it is very necessary to propose an intraoperative fast planning system and method for ablation therapy, which can correct the incomplete ablation problem that may occur due to the deviation of the actual probe position during the operation from the preoperative planned probe position before operation.

### SUMMARY OF THE INVENTION

An object of the application is to provide an intraoperative fast planning system and method for ablation therapy, which solves the problem of incomplete ablation that may occur due to the deviation of the actual probe position during ablation operation from the preoperative planned probe position, and is beneficial to realize accurate control of the thermal dosage in focus area and ensure effectiveness of the ablation.

In order to solve the above problems, the present application discloses an intraoperative fast planning system for ablation therapy, comprising:

an input device, configured to obtain a boundary of a tumor and an actual position of an ablation probe relative to the tumor, a quantitative relationship between an ablation range and ablation parameters of the ablation probe, and maximum ablation range parameters of the ablation probe.

a calculation device, configured to calculate a target treatment area according to the boundary of the tumor and the actual position of the ablation probe relative to the tumor, and determine whether the maximum ablation range of the ablation probe can cover the target treatment area in combination with the maximum ablation range parameters, wherein if the maximum ablation range can cover the target treatment area, calculate ablation parameters for the ablation probe according to the actual position of the ablation probe and the target treatment area as well as the quantitative relationship between the ablation range and the ablation parameters of the ablation probe; and

an output device, configured to output the ablation parameters.

In an embodiment, the calculation device is further configured to output information indicating a need to supplement probe or a change in the position of the probe via the output device if the maximum ablation range cannot cover the target treatment area.

In an embodiment, the calculation device is further configured to calculate a supplementary probe position or a changed probe position according to the target treatment area if the maximum ablation range cannot cover the target treatment area, and output the supplementary probe position or the changed probe position via the output device.

In an embodiment, the input device comprises a first interface coupled to an external imaging device and the first interface is configured to receive image data from the external imaging device;

wherein the input device also comprises an image analysis module which is configured to perform image analysis on the image data to obtain the boundary of the tumor and the actual position of the ablation probe relative to the tumor.

In an embodiment, the external imaging device is an X-ray machine or a CT machine or an MRI machine or an ultrasound machine.

In an embodiment, the input device comprises one of the following devices or any combination thereof: a keyboard, a mouse, or a touch screen.

In an embodiment, the output device comprises a monitor which is configured to display the ablation parameters.

In an embodiment, the output device comprises a second interface coupled to an ablation control device, the second is configured to output the ablation parameters to the ablation control device for use during an ablation process.

In an embodiment, the calculation device calculates the ablation parameters of the ablation probe according to the following quantitative relationship between the ablation range and the ablation parameters of the ablation probe:

Xₚ=Yₚ=c₁c₂×c₃^{t}

Zₚ=c₄-c₅×c₆^{t}

wherein, c₁, c₂, c₃, c₄, c₅, c₆, c₇ are fixation factors related to the type of the ablation probe, and t is time.

In an embodiment, the calculation device calculates the ablation parameters of the ablation probe according to the following quantitative relationship between the ablation range and the ablation parameters of the ablation probe:

Xₚ=Yₚ=c₇×t+c₈

Zₚ=c₉×t+c₁₀

wherein, c₇, c₈, c₉, c₁₀ are fixation factors related to the type of the ablation probe, and t is time.

In an embodiment, the calculation device calculates the ablation parameters of the ablation probe according to the following quantitative relationship between the ablation range and the ablation parameters of the ablation probe:

Xₚ=Yₚ=c₁₁×exp(−t/c₁₂)+c₁₃

Zₚ=c₁₄×exp(−t/c₁₅)+c₁₆

wherein, c₁₁, c₁₂, c₁₃, c₁₄, c₁₅, c₁₆ are fixation factors related to the type of the ablation probe, and t is time.

In an embodiment, the ablation probe comprises an unipolar, a bipolar and a multipolar ablation probe.

In an embodiment, the quantitative relationship of the ablation parameters comprises a function of the ablation range versus power and time, a function of the ablation range versus input energy, and a function of the ablation range versus center temperature.

In an embodiment, safety boundary of the maximum ablation range is 5 mm beyond the boundary of the tumor, or any distance beyond the boundary of the tumor according to the doctor's advice.

In an embodiment, the ablation comprises but not limited to radiofrequency ablation, cryoablation, microwave ablation, and multi-modal ablation.

The application also discloses an intraoperative fast planning method for ablation therapy, comprising:

obtaining a boundary of the tumor and an actual position of an ablation probe relative to a tumor;

obtaining a quantitative relationship between the ablation range and the ablation parameters of the ablation probe;

obtaining maximum ablation range parameters of the ablation probe;

calculating a target treatment area according to the boundary of the tumor and the actual position of the ablation probe relative to the tumor, and determining whether the maximum ablation range of the ablation probe can cover the target treatment area in combination with the maximum ablation range parameters; and

if the maximum ablation range can cover the target treatment area, calculating ablation parameters for the ablation probe according to the actual position of the ablation probe and the target treatment area as well as the quantitative relationship between the ablation range and the ablation parameters of the ablation probe, and outputting the ablation parameters.

In an embodiment, the method further comprises outputting information indicating a need to supplement probe or a change in the position of the probe via the output device if the maximum ablation range cannot cover the target treatment area.

In an embodiment, the method further comprises calculating a supplementary probe position or a changed probe position according to the target treatment area if the maximum ablation range cannot cover the target treatment area, and outputting the supplementary probe position or the changed probe position via the output device.

In an embodiment, the method further comprises obtaining image data from an external imaging device via an input device and performing image analysis on the image data to obtain the boundary of the tumor and the actual position of the ablation probe relative to the tumor.

In an embodiment, the external imaging device is an X-ray machine or a CT machine or an MRI machine or an ultrasound machine.

In an embodiment, the input device comprises one of the following devices or any combination thereof: a keyboard, a mouse, or a touch screen.

In an embodiment, the output device comprises a monitor which is configured to display the ablation parameters.

In an embodiment, outputting the ablation parameters to the ablation control device via the output device for use during an ablation process.

In an embodiment, the quantitative relationship of the ablation parameters comprises a function of the ablation range versus power and time, a function of the ablation range versus input energy, and a function of the ablation range versus center temperature.

In an embodiment, the calculation device calculates the ablation parameters of the ablation probe according to the following quantitative relationship between the ablation range and the ablation parameters of the ablation probe:

Xₚ=Yₚ=c₁-c₂×c₃^{t}

Zₚ=c₄-c₅×c₆^{t}

wherein, c₁, c₂, c₃, c₄, c₅, c₆, c₇ are fixation factors related to the type of the ablation probe, and t is time.

In an embodiment, the calculation device calculates the ablation parameters of the ablation probe according to the following quantitative relationship between the ablation range and the ablation parameters of the ablation probe:

Xₚ=Yₚ=c₇×t+c₈

Zₚ=c₉×t+c₁₀

wherein, c₇, c₈, c₉, c₁₀ are fixation factors related to the type of the ablation probe, and t is time.

In an embodiment, the calculation device calculates the ablation parameters of the ablation probe according to the following quantitative relationship between the ablation range and the ablation parameters of the ablation probe:

Xₚ=Yₚ=c₁₁×exp(−t/c₁₂)+c₁₃

Zₚ=c₁₄×exp(−t/c₁₅)+c₁₆

wherein, c₁₁, c₁₂, c₁₃, c₁₄, c₁₅, c₁₆ are fixation factors related to the type of the ablation probe, and t is time.

In an embodiment, the ablation probe comprises an unipolar, a bipolar and a multipolar ablation probe.

In an embodiment, safety boundary of the maximum ablation range is 5 mm beyond the boundary of the tumor, or any distance beyond the boundary of the tumor according to the doctor's advice.

In an embodiment, the ablation comprises but not limited to radiofrequency ablation, cryoablation, microwave ablation, and multi-modal ablation.

The application also discloses an intraoperative fast planning system for ablation therapy, comprising:

an input device, configured to obtain a boundary of the tumor and an actual position of an ablation probe relative to a tumor, a quantitative relationship between an ablation range and ablation parameters of the ablation probe, and maximum ablation range parameters of the ablation probe.

an output device, configured to output the ablation parameters;

a memory, configured to store computer-executable instructions; and

a processor, configured to implement the steps in the method described above when executing the computer-executable instructions.

The application also discloses a computer-readable storage medium the computer-readable storage medium stores computer-executable commands which are executed by a processor to implement the steps in the method described above.

Compared with the prior art, the embodiments of the present application have at least the following differences and effects:

To solve the problem of incomplete ablation that may occur due to the deviation of the actual probe position during ablation operation from the preoperative planned probe position in the prior art, the application provides an intraoperative fast planning system and method for ablation therapy, which are beneficial to realize accurate control of the thermal dosage in focus area and ensure effectiveness of the ablation.

A large number of technical features are described in the specification of the present application, and are distributed in various technical solutions. If a combination (i.e., a technical solution) of all possible technical features of the present application is listed, the description may be made too long. In order to avoid this problem, the various technical features disclosed in the above summary of the present application, the technical features disclosed in the various embodiments and examples below, and the various technical features disclosed in the drawings can be freely combined with each other to constitute various new technical solutions (all of which are considered to have been described in this specification), unless a combination of such technical features is not technically feasible. For example, feature A+B+C is disclosed in one example, and feature A+B+D+E is disclosed in another example, while features C and D are equivalent technical means that perform the same function, and technically only choose one, not to adopt at the same time. Feature E can be combined with feature C technically. Then, the A+B+C+D scheme should not be regarded as already recorded because of the technical infeasibility, and A+B+C+E scheme should be considered as already documented.

### BRIEF DESCRIPTION OF THE FIGURES

FIG. 1 is a schematic block diagram of an intraoperative fast planning system for ablation therapy according to a first embodiment of the present application.

FIG. 2 is a schematic flowchart of an intraoperative fast planning method for ablation therapy according to a second embodiment of the present application.

FIG. 3 is a schematic flowchart of an embodiment of step 204 to step 205 according to the second embodiment of the present application.

FIG. 4 is a schematic diagram of definitions of X, Y, Z directions and ablation range of an ablation probe according to an embodiment of the present application.

FIG. 5 is an illustration of deviation of the insertion position from the tumor center when an ablation probe is actually inserted according to an embodiment of the present application.

FIG. 6 is an illustration of a required ablation range defined according to an actual probe position according to an embodiment of the present application.

### LIST OF REFERENCE SIGNS

101—Input device
102—Calculation device
103—Output device
104—External imaging device
105—Ablation control device
1011—Image analysis module

### DETAILED DESCRIPTION

In the following description, numerous technical details are set forth in order to provide the reader with a better understanding of the present application. However, those skilled in the art can understand that the technical solutions claimed in the present application can be implemented without these technical details and various changes and modifications based on the following embodiments.

The ablation techniques applicable to various embodiments of the present application comprise, but not limited to, radiofrequency ablation, cryoablation, microwave ablation, multi-modal ablation, and so on. Since the multi-modal ablation requires more precise thermal dosage control, in the following, the implementation of the present application will be further described in detail with reference to the accompanying drawings and taking multi-modal ablation as an example, so as to enhance the understanding of the purpose, technical solutions and advantages of the present application.

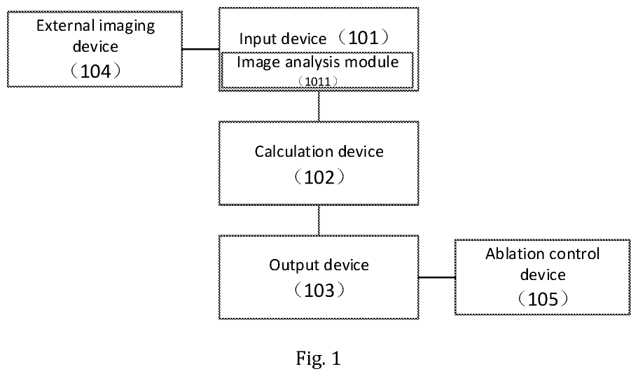

A first embodiment of the present application discloses an intraoperative fast planning system for ablation therapy, the structure of which is shown in FIG. 1. The intraoperative fast planning system for ablation therapy comprises an input device 101, an output device 103 and a calculation device 102.

Typically:

(1) The input device 101 is configured to obtain a boundary of the tumor and an actual position of an ablation probe relative to a tumor, a quantitative relationship between an ablation range and ablation parameters of the ablation probe, and maximum ablation range parameters of the ablation probe. Optionally, the input device 101 comprises a first interface coupled to an external imaging device 104, and the input device 101 obtains image data from the external imaging device 104 via the first interface. Optionally, the input device 101 further comprises an image analysis module 1011, the image analysis module 1011 is configured to perform image analysis on the obtained image data to obtain the boundary of the tumor and the actual position of the ablation probe relative to the tumor. Optionally, the external imaging device 104 comprises at least an X-ray machine or a CT machine or a MRI machine or an ultrasound machine or other medical imaging device. Optionally, the input device 101 comprises at least one of the following devices or any combination thereof: a keyboard, a mouse, a touch screen.

The maximum ablation range parameters of the ablation probe can be obtained in various ways. Optionally, they may be obtained by querying a database or a configuration table according to the specification of the ablation probe. Optionally, they may be directly input or selected from a list via an input device 101 (such as a keyboard and mouse, and so on).

Optionally, the specification of the ablation probe may be an unipolar, a bipolar or a multipolar ablation probe or the like.

(2) The calculation device 102 is configured to calculate a target treatment area according to the boundary of the tumor and the actual position of the ablation probe relative to the tumor, and determine whether the maximum ablation range of the ablation probe can cover the target treatment area in combination with the maximum ablation range parameters. If the maximum ablation range can cover the target treatment area, calculates ablation parameters for the ablation probe according to the actual position of the ablation probe and the target treatment area as well as the quantitative relationship between the ablation range and the ablation parameters of the ablation probe, and outputs the ablation parameters. Wherein, the target treatment area is the target treatment area covering the tumor and an ablation safety boundary required in three-dimensional space. Optionally, the calculation device 102 is further configured to output information indicating a need to supplement probe or a change in the position of the probe via the output device 103 if the maximum ablation range cannot cover the target treatment area. Optionally, the calculation device 102 is further configured to calculate a supplementary probe position or a changed probe position according to the target treatment area if the maximum ablation range cannot cover the target treatment area, and output the supplementary probe position or the changed probe position via the output device 103.

Optionally, there are multiple methods for determining “whether the maximum ablation range of the ablation probe can cover the target treatment area” by the calculation device. In an embodiment, the calculation device first obtains the image data after the actual probe insertion, defines directions perpendicular to the probe insertion direction as X and Y direction, defines direction parallel to the probe insertion direction as Z direction, and defines an ablation center point on the ablation probe as an origin O (xₒ, yₒ, zₒ). Then, the calculation device obtains the actual probe position of the ablation probe, coordinates (xᵢ, yᵢ, zᵢ) of each point of the boundary of the tumor after considering the ablation safety boundary in the same coordinate system, and the actual position of the ablation probe relative to the tumor, and calculates the target treatment area range Xₜ, Yₜ, Zₜ covering the tumor and the ablation safety boundary required in the X, Y and Z directions (wherein, 2 times of the maximum distance from each point of the boundary of the tumor to YOZ plane in cross section is Xₜ, that is, Xₜ=max|xᵢ-xₒ|, 2 times of the maximum distance from each point of the boundary of the tumor to XOY plane in cross section is Zₜ, that is, Zₜ=max|zᵢ-zₒ|, 2 times of the maximum distance from each point of the boundary of the tumor to XOZ plane on sagittal plane is Yₜ, that is, Yₜ=max|yᵢ-yₒ|). Finally, matching the maximum ablation range Xₘₐₓ, Yₘₐₓ, Zₘₐₓ of this specification of the probe in X, Y and Z directions with the size Xₜ, Yₜ, Zₜ, if satisfies condition “Xₘₐₓ≥Xₜ, Yₘₐₓ≥Yₜ, Zₘₐₓ≥Zₜ”, the calculation device determines that the maximum ablation range can cover the target treatment area; otherwise, the calculation device determines that the maximum ablation range cannot cover the target treatment area. The “ablation safety boundary” is preset. Optionally, the ablation safety boundary is (5-10) mm beyond the boundary of the tumor, preferably 5 mm. Optionally, the ablation safety boundary is any distance beyond the boundary of the tumor according to the doctor's advice.

Optionally, the calculation device 102 first establishes a quantitative relationship between the ablation range and the ablation parameters of the ablation probe, and then calculates the ablation parameters of the ablation probe according to the quantitative relationship established first, and then calculates the ablation parameters of the ablation probe according to the quantitative relationship, the actual position of the ablation probe and the target treatment area. The quantitative relationship can be configured to predict the treatment temperature, thermal dosage and damage range. The quantitative relationship may be linear or nonlinear. There are multiple choices for functional relationship composition of the quantitative relationship. Optionally, the quantitative relationship is a function of the ablation range versus the power and the time. Optionally, the quantitative relationship is a function of the ablation range versus the input energy or the center temperature.

In an embodiment of “calculation device 102 establishes a quantitative relationship between the ablation range and the ablation parameters of the ablation probe.”, establishes a multi-modal ablation theoretical model according to the ablation probe in consideration of changes in tissue physical properties after frozen; then, simulates the temperature field in finite element simulation software Comsol 5.2, and calculates the quantitative relationship between the ablation range and the ablation parameters in the direction parallel to the probe direction (Z direction) and directions perpendicular to the probe direction (X and Y directions), and the maximum ablation range Xₘₐₓ, Yₘₐₓ, Zₘₐₓ in the X, Y and Z directions corresponding to the type of the probe. FIG. 4 is a schematic diagram of the definition of X, Y and Z directions and the ablation range of the probe. Because for the ablation probe, uses the method of energy control (the power is always kept constant) clinically to perform surgery. The input energy is controlled by changing the length of the ablation time, thereby changing the ablation range, so finally obtains the relationship between the ablation range and the ablation time of the probe. Since the ablation range of the ablation probe is symmetric, the quantitative relationship between the ablation range in the X direction and the Y direction is the same.

In a specific embodiment, the quantitative relationship between the ablation range and the ablation parameters of the ablation probe comprises formulas \textcircled{1} and \textcircled{2}, wherein, c₁, c₂, c₃, c₄, c₅, c₆, c₇ are constants related to the type of the ablation probe, and t is time.

Xₚ=Yₚ=c₁-c₂×c₃^{t} \textcircled{1}

Zₚ=c₄-c₅×c₆^{t} \textcircled{2}

In a specific embodiment, the quantitative relationship between the ablation range and the ablation parameters of the ablation probe comprises formulas \textcircled{3} and \textcircled{4}, wherein, c₇, c₈, c₉, c₁₀ are constants related to the type of the ablation probe, and t is time.

Xₚ=Yₚ=c₇×t+c₈ \textcircled{3}

Zₚ=c₉×t+c₁₀ \textcircled{4}

In a specific embodiment, the quantitative relationship between the ablation range and the ablation parameters of the ablation probe comprises formulas \textcircled{5} and \textcircled{6}, wherein, c₁₁, c₁₂, c₁₃, c₁₄, c₁₅, c₁₆ are constants related to the type of the ablation probe, and t is time.

Xₚ=Yₚ=c₁₁×exp(−t/c₁₂)+c₁₃ \textcircled{5}

Zₚ=c₁₄×exp(−t/c₁₅)+c₁₆ \textcircled{6}

Optionally, the calculation device 102 calculates the ablation parameters satisfying the ablation requirements according to a gradient method, a look-up table method or a neural network method.

(3) The output device 103 is configured to output the ablation parameters. Optionally, the output device 103 comprises a monitor which is configured to display the ablation parameters, and the prompt information indicating a need to supplement probe, and so on. Optionally, the output device 103 comprises a second interface coupled to an ablation control device 105, the second interface is configured to output the ablation parameters to the ablation control device 105 for use by the ablation control device 105 during an ablation process.

A second embodiment is a method embodiment corresponding to the first embodiment. The technical details of the first embodiment can also be applied to the second embodiment, and the technical details of the second embodiment can be applied to the first embodiment.

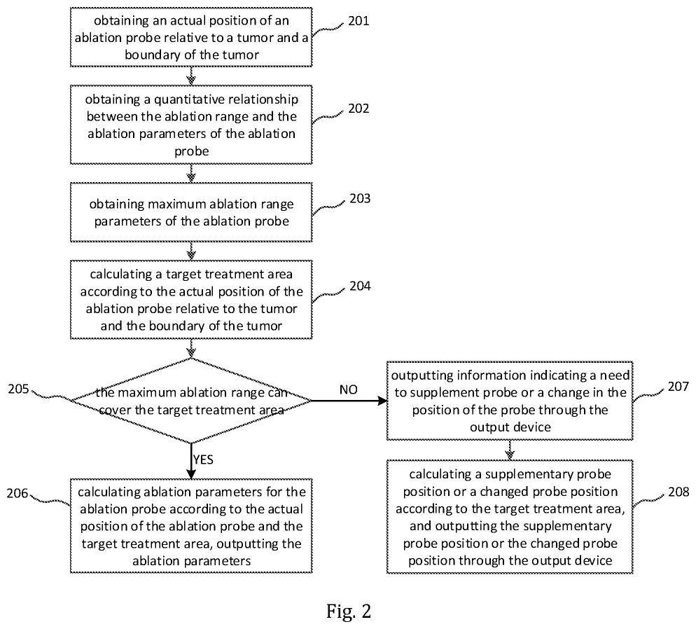

The second embodiment of the present application discloses an intraoperative fast planning method for multi-modal ablation therapy, the flowchart of which is shown in FIG. 2, and the method comprises the following steps:

Firstly, performing step 201: obtaining a boundary of the tumor and an actual position of the ablation probe relative to the tumor. Optionally, the input device 101 obtains image data from an external imaging device 104 and analyzes the image data to obtain the boundary of the tumor and the actual position of the ablation probe relative to the tumor. Optionally, the external imaging device 104 comprises at least an X-ray machine or a CT machine or a MRI machine or an ultrasound machine or other medical imaging device. Optionally, the input device 101 comprises at least one of the following devices or any combination thereof: a keyboard, a mouse, a touch screen.

Optionally, before performing the step 201, the method also comprises obtaining specifications and fixation parameter information of the ablation probe. Optionally, the specification of the ablation probe may be a unipolar, a bipolar or a multipolar ablation probe or the like.

Thereafter, performing step 202: obtaining quantitative relationship between an ablation range and ablation parameters of the ablation probe. The quantitative relationship can be configured to predict the treatment temperature, thermal dosage and damage range. The quantitative relationship may be linear or nonlinear. There are multiple choices for functional relationship composition of the quantitative relationship. Optionally, the quantitative relationship is a function of the ablation range versus the power and the time. Optionally, the quantitative relationship is a function of the ablation range versus the input energy or the center temperature.

Thereafter, performing step 203, obtaining maximum ablation range parameters of the ablation probe. The maximum ablation range parameters of the ablation probe are determined by the type of the selected ablation probe and the type of the tissue to be ablated, the maximum ablation range parameters are the same for the same type of the ablation probe and the same type of the tissue to be ablated, and the maximum ablation range parameters are different for different type of the ablation probe or different types of the tissue to be ablated.

The maximum ablation range parameters of the ablation probe can be obtained in various ways. Optionally, they may be obtained by querying a database or a configuration table according to the specifications of the ablation probe. Optionally, they may be directly input or selected from a list via an input device 101 (such as a keyboard and mouse, and so on). Thereafter, performing step 204: calculating a target treatment area according to the boundary of the tumor and the actual position of the ablation probe relative to the tumor.

Thereafter, performing step 205, determining whether satisfies the condition “the maximum ablation range of the ablation probe can cover the target treatment area”, and performing step 206 if satisfies the condition.

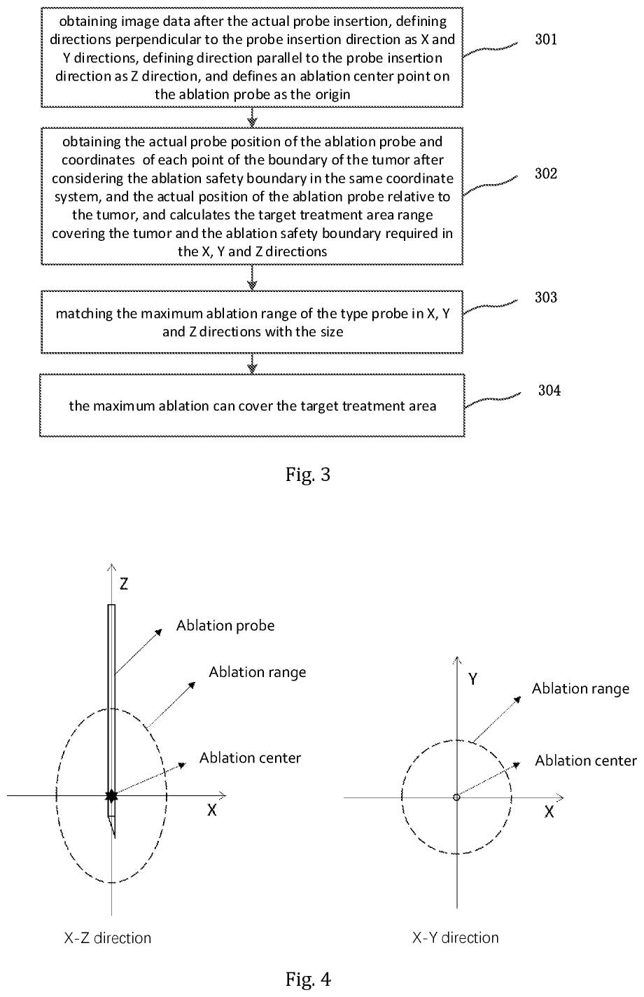

As shown in FIG. 3, an embodiment of a combination of the step 204 and the step 205 specifically comprises the following sub-steps:

Firstly, preforming step 301, obtaining image data after the actual probe insertion, defining directions perpendicular to the probe insertion direction as X and Y directions, defining direction parallel to the probe insertion direction as Z direction, and defines an ablation center point on the ablation probe as the origin O (xₒ, yₒ, zₒ);

Then, performing step 302: obtaining the actual probe position of the ablation probe and coordinates (xᵢ, yᵢ, zᵢ) of each point of the boundary of the tumor after considering the ablation safety boundary in the same coordinate system, and the actual position of the ablation probe relative to the tumor, and calculates the target treatment area range Xₜ, Yₜ, Zₜ covering the tumor and the ablation safety boundary required in the X, Y and Z directions (wherein, 2 times of the maximum distance from each point of the boundary of the tumor to YOZ plane in cross section is Xₜ, that is, Xₜ=max|xᵢ-xₒ|, 2 times of the maximum distance from each point of the boundary of the tumor to XOY plane in cross section is Zₜ, that is, Zₜ=max|zᵢ-zₒ|, 2 times of the maximum distance from each point of the boundary of the tumor to XOZ plane on sagittal plane is Yₜ, that is, Yₜ=max|yᵢ-yₒ|).

Then, performing step 303: matching the maximum ablation range of the type probe in X, Y and Z directions Xₘₐₓ, Yₘₐₓ, Zₘₐₓ with the size Xₜ, Yₜ, Zₜ;

Then, performing step 304, determining whether the maximum ablation can cover the target treatment area, or in other words, whether satisfies the condition “Xₘₐₓ≥Xₜ, Yₘₐₓ≥Yₜ, Zₘₐₓ≥Zₜ”. the calculation device determines that the maximum ablation range can cover the target treatment area; otherwise, the calculation device determines that the maximum ablation range cannot cover the target treatment area.

Furthermore, if satisfies the condition “Xₘₐₓ≥Xₜ, Yₘₐₓ≥Yₜ, Zₘₐₓ≥Zₜ”. then performing step 206, if not satisfies the condition “Xₘₐₓ≥Xₜ, Yₘₐₓ≥Yₜ, Zₘₐₓ≥Zₜ”, performing step 207 until the end.

The “ablation safety boundary” is preset. Optionally, the ablation safety boundary is (5-10) mm beyond the boundary of the tumor, preferably 5 mm. Optionally, the ablation safety boundary is any distance beyond the boundary of the tumor according to the doctor's advice.

Finally, performing step 206: calculating ablation parameters for the ablation probe according to the actual position of the ablation probe and the target treatment area, and outputting the ablation parameters.

Optionally, the step 206 further comprises first establishing a quantitative relationship between the ablation range and the ablation parameters of the ablation probe, and then calculating the ablation parameters of the ablation probe according to the quantitative relationship, the actual position of the ablation probe and the target treatment area. The quantitative relationship can be configured to predict the treatment temperature, thermal dosage and damage range. The quantitative relationship may be linear or nonlinear. There are multiple choices for functional relationship composition of the quantitative relationship. Optionally, the quantitative relationship is a function of the ablation range versus the power and the time. Optionally, the quantitative relationship is a function of the ablation range versus the input energy or the center temperature.

In an embodiment of “establishing a quantitative relationship between the ablation range and the ablation parameters of the ablation probe.”, establishing a multi-modal ablation theoretical model according to the ablation probe in consideration of changes in tissue physical properties after froze; then, simulating the temperature field in finite element simulation software Comsol 5.2, and calculating the quantitative relationship between the ablation range and the ablation parameters in the direction parallel to the probe direction (Z direction) and directions perpendicular to the probe direction (X and Y directions), and the maximum ablation range Xₘₐₓ, Yₘₐₓ, Zₘₐₓ in the X, Y and Z directions corresponding to the type of the probe. FIG. 1 is a schematic diagram of the definition of X, Y and Z directions and the ablation range of the probe. Because for the ablation probe, uses the method of energy control (the power is always kept constant) clinically to perform surgery. The input energy is controlled by changing the length of the ablation time, thereby changing the ablation range, so finally obtains the relationship between the ablation range and the ablation time of the probe. Since the ablation range of the ablation probe is symmetric, the quantitative relationship between the ablation range in the X direction and the Y direction is the same.

In a specific embodiment, the quantitative relationship between the ablation range and the ablation parameters of the ablation probe comprises formulas \textcircled{1} and \textcircled{2}, wherein, c₁, c₂, c₃, c₄, c₅, c₆, c₇ are constants related to the type of the ablation probe, and t is time.

Xₚ=Yₚ=c₁-c₂×c₃^{t} \textcircled{1}

Zₚ=c₄-c₅×c₆^{t} \textcircled{2}

In a specific embodiment, the quantitative relationship between the ablation range and the ablation parameters of the ablation probe comprises formulas \textcircled{3} and \textcircled{4}, wherein, c₇, c₈, c₉, c₁₀ are constants related to the type of the ablation probe, and t is time.

Xₚ=Yₚ=c₇×t+c₈ \textcircled{3}

Zₚ=c₉×t+c₁₀ \textcircled{4}

In a specific embodiment, the quantitative relationship between the ablation range and the ablation parameters of the ablation probe comprises formulas \textcircled{5} and \textcircled{6}, wherein, c₁₁, c₁₂, c₁₃, c₁₄, c₁₅, c₁₆ are constants related to the type of the ablation probe, and t is time.

Xₚ=Yₚ=c₁₁×exp(−t/c₁₂)+c₁₃ \textcircled{5}

Zₚ=c₁₄×exp(−t/c₁₅)+c₁₆ \textcircled{6}

As mentioned above, the calculation method of the ablation parameters satisfying the ablation requirements may be a gradient method, a look-up table method or a neural network method.

Optionally, the method also comprises step 207: outputting information indicating a need to supplement probe or a change in the position of the probe via the output device 103 if the maximum ablation range cannot cover the target treatment area, i.e., “the maximum ablation range of the ablation probe cannot cover the target treatment area”. Optionally, after the step 207, performing step 208, calculating a supplementary probe position or a changed probe position according to the target treatment area, and outputting the supplementary probe position or the changed probe position via the output device 103. Optionally, after the step 208, re-selecting the ablation probe, and then performing the steps 201-205 and 206 again.

In order to better understand the technical solutions of this specification, the following description will be given with a specific embodiment. The details listed in this embodiment are mainly for ease of understanding and are not intended to limit the scope of protection of this application.

The embodiment takes a bipolar ablation probe (the type is 3 cm) as an example, and comprises the following steps:

S1: establishing a multi-modal ablation theoretical model according to the ablation probe in consideration of changes in tissue physical properties after froze; then, simulating the temperature field in finite element simulation software Comsol 5.2, and calculating the quantitative relationship between the ablation range and the ablation parameters in the direction parallel to the probe direction (Z direction) and directions perpendicular to the probe direction (X and Y directions), and the maximum ablation range Xₘₐₓ, Yₘₐₓ, Zₘₐₓ in the X, Y and Z directions corresponding to the type of the probe. FIG. 4 is a schematic diagram of the definition of X, Y and Z directions and the ablation range of the probe. Because for the ablation probe, uses the method of energy control (the power is always kept constant) clinically to perform surgery. The input energy is controlled by changing the length of the ablation time, thereby changing the ablation range, so finally obtains the relationship between the ablation range and the ablation time of the probe. Since the ablation range of the ablation probe is symmetric, the quantitative relationship between the ablation range in the X direction and the Y direction is the same

Xₚ=Yₚ=c₁-c₂×c₃^{t}

Zₚ=c₄-c₅×c₆^{t}

or:

Xₚ=Yₚ=c₇×t+c₈

Zₚ=C₉×t+C₁₀

or:

Xₚ=Yₚ=c₁₁×exp(−t/c₁₂)+c₁₃

Zₚ=c₁₄×exp(−t/c₁₅)+c₁₆

Wherein, c_{1˜16} are constant, and t is time.

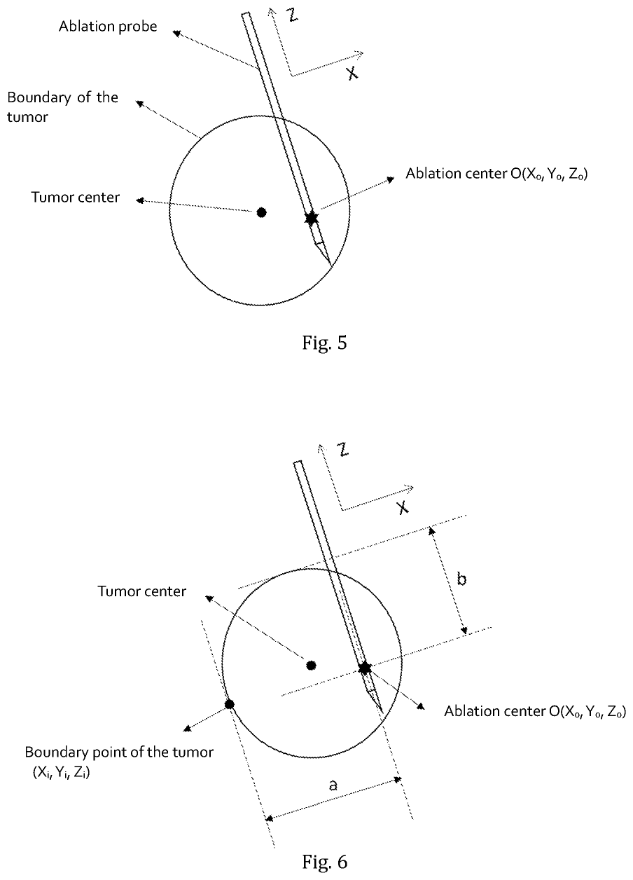

S2: reading image data after the actual probe insertion, defining directions perpendicular to the probe insertion direction as X and Y directions, defining direction parallel to the probe insertion direction as Z direction, and defining the ablation center point on the ablation probe as the origin O (xₒ, yₒ, zₒ), as shown in FIG. 5.

Obtaining the actual probe position of the ablation probe, coordinates (xᵢ, yᵢ, zᵢ) of each point of the boundary of the tumor after considering the ablation safety boundary in the same coordinate system, and the actual position of the ablation probe relative to the tumor, and calculating the target treatment area range Xₜ, Yₜ, Zₜ covering the tumor and the ablation safety boundary required in the X, Y and Z directions. As shown in FIG. 6, wherein, 2 times of the maximum distance from each point of the boundary of the tumor to YOZ plane in cross section is xₜ, that is, Xₜ=max|xᵢ-xₒ|, 2 times of the maximum distance from each point of the boundary of the tumor to XOY plane in cross section is zₜ, that is, Zₜ=max|zᵢ-zₒ|, 2 times of the maximum distance from each point of the boundary of the tumor to XOZ plane on sagittal plane is Yₜ, that is, Yₜ=max|yᵢ-yₒ|.

Xₜ=2a

Yₜ=2c

Zₜ=2b

S3, matching the maximum ablation range Xₘₐₓ, Yₘₐₓ, Zₘₐₓ of the type probe in X, Y and Z directions with the size Xₜ, Yₜ, Zₜ, if Xₘₐₓ≥Xₜ, Yₘₐₓ≥Yₜ, Zₘₐₓ≥Zₜ, calculating ablation parameters that satisfy the ablation requirements according to quantitative relationship between the ablation range Xₚ, Yₚ, Zₚ and the ablation parameters, and the size Xₜ, Yₜ, Zₜ, and outputting the currently planned ablation parameters. In the embodiment, only need to substitute Xₜ, Yₜ, Zₜ into the quantitative relationship to calculate the ablation time respectively, and then select the largest ablation time to satisfy the requirements of the ablation. Otherwise, it is suggested to increase the number of the ablation probes, and re-plan and calculate.

It should be noted that those skilled in the art will understand that the implementation functions of each module shown in the implementation method of the above multi-modal ablation fast planning system can be understood by referring to the relevant description of the above multi-modal ablation fast planning method. The functions of each module shown in the above embodiments of the intraoperative planning system for multi-modal ablation therapy can be implemented by a program (executable instructions) running on a processor, or by a specific logic circuit. If the intraoperative planning system for multi-modal ablation therapy described above is implemented in the form of a software function module and sold or used as an independent product, it may also be stored in a computer-readable storage medium. Based on the understanding, the technical solutions of the embodiments of the present invention in essence or part of contributions to the prior art can be embodied in the form of software products. The computer software product is stored in a storage medium, and comprises several instructions to enable a computer device (which may be a personal computer, server, or network device, etc.) to perform all or part of the methods described in the embodiments of the present invention. The foregoing storage media comprise various media that can store program codes, such as a U disk, a mobile hard disk, a read-only memory (ROM, Read Only Memory), a magnetic disk, or an optical disk. In this way, the embodiments of the present invention are not limited to any specific combination of hardware and software.

Correspondingly, an embodiment of the present invention also discloses a computer storage medium in which computer-executable instructions are stored. When the computer-executable instructions are executed by a processor, the method embodiments of the present application are implemented. The computer-readable storage media comprises permanent and non-permanent, removable and non-removable media, information storage can be achieved by any method or technology. Information can be computer-readable instructions, data structures, modules of programs, or other data. Examples of computer-readable storage media comprise, but not limited to, Phase Change Memory (PRAM), Static Random Access Memory (SRAM), Dynamic Random Access Memory (DRAM), other types of Random Access Memory (RAM), Read Only Memory (ROM), Electrically Erasable Programmable Read Only Memory (EEPROM), Flash Memory or other Memory Technology, Read Only Disc, Read Only Memory (CD-ROM), Digital Versatile Disc (DVD) or other optical storage, Magnetic Tape Cassette, Magnetic tape magnetic disk storage or other magnetic storage device or any other non-transmission medium that can be used to store information that can be accessed by the calculation device. As defined herein, computer-readable storage media do not comprise temporary computer-readable media, such as modulated data signals and carrier waves.

In addition, an embodiment of the application also discloses an intraoperative fast planning system for multi-modal ablation therapy, comprising an input device configured to obtain an actual position of an ablation probe relative to a tumor, a boundary of the tumor and maximum ablation range parameters of the ablation probe, an output device configured to output the ablation parameters, a memory configured to store computer-executable instructions, and a processor configured to implement the steps in the method embodiments described above when executing the computer-executable instructions stored in the memory. Wherein, the processor may be a Central Processing Unit (CPU), other general-purpose processors, Digital Signal Processor (DSP), Application Specific Integrated Circuit (ASIC), etc. The memory may be read-only memory (ROM), random access memory (RAM), flash memory (Flash), hard disk or solid-state disk, etc. The steps of the method disclosed in each embodiment of the application can be directly embodied as the execution of the hardware processor or the combined execution of the hardware and software modules in the processor.

It should be noted that in the application documents of the present patent, relational terms such as first and second, and so on are only configured to distinguish one entity or operation from another entity or operation, and do not necessarily require or imply any such actual relationship or order between these entities or operations. Furthermore, the term “comprises” or “comprising” or “includes” or any other variations thereof is intended to encompass a non-exclusive inclusion, such that a process, method, article, or device that comprises a plurality of elements includes not only those elements but also other elements, or elements that are inherent to such a process, method, item, or device. Without more restrictions, the element defined by the phrase “comprise(s) a/an” does not exclude that there are other identical elements in the process, method, item or device that includes the element. In the application file of this patent, if it is mentioned that an action is performed according to an element, it means the meaning of performing the action at least according to the element, and includes two cases: the behavior is performed only on the basis of the element, and the behavior is performed based on the element and other elements. Multiple, repeatedly, various, etc., expressions include 2, twice, 2 types, and 2 or more, twice or more, and 2 types or more types.

All documents mentioned in the application are considered to be included in the application of the disclosure as a whole, so that they can be used as a basis for modification when necessary. In addition, it should be understood that the above descriptions are only preferred embodiments of this specification, and are not intended to limit the protection scope of this specification. Any modification, equivalent replacement, improvement, etc. made within the spirit and principle of one or more embodiments of this specification should be included in the protection scope of one or more embodiments of this specification.

Specific embodiments of this specification have been described above. Other embodiments are within the scope of the appended claims. In some cases, the actions or steps described in the claims can be performed in a different order than in the embodiments and still achieve desired results. In addition, the processes depicted in the drawings do not necessarily require the specific order or sequential order shown in order to achieve the desired results. In some embodiments, multitasking and parallel processing are also possible or may be advantageous.

1. An intraoperative fast planning system for ablation therapy, comprising:
an input device, configured to obtain a boundary of a tumor and an actual position of an ablation probe relative to the tumor, a quantitative relationship between an ablation range and ablation parameters of the ablation probe, and maximum ablation range parameters of the ablation probe.
a calculation device, configured to calculate a target treatment area according to the boundary of the tumor and the actual position of the ablation probe relative to the tumor, and determine whether the maximum ablation range of the ablation probe can cover the target treatment area in combination with the maximum ablation range parameters, wherein if the maximum ablation range can cover the target treatment area, then calculating ablation parameters for the ablation probe according to the actual position of the ablation probe and the target treatment area as well as the quantitative relationship between the ablation range and the ablation parameters of the ablation probe; and
an output device, configured to output the ablation parameters.

2. The system according to claim 1, wherein the calculation device is further configured to output information indicating a need to supplement probe or a change in the position of the probe via the output device if the maximum ablation range cannot cover the target treatment area.

3. The system according to claim 1, wherein the calculation device is further configured to calculate a supplementary probe position or a changed probe position according to the target treatment area if the maximum ablation range cannot cover the target treatment area, and output the supplementary probe position or the changed probe position via the output device.

4. The system according to claim 1, wherein the input device comprises a first interface coupled to an external imaging device, and the first interface is configured to receive image data from the external imaging device;
the input device also comprises an image analysis module which is configured to perform image analysis on the image data to obtain the boundary of the tumor and the actual position of the ablation probe relative to the tumor.

5. The system according to claim 4, wherein the external imaging device is an X-ray machine or a CT machine or an MRI machine or an ultrasound machine.

6. The system according to claim 1, wherein the input device comprises one of the following devices or any combination thereof: a keyboard, a mouse, or a touch screen.

7. The system according to claim 1, wherein the output device comprises a monitor which is configured to display the ablation parameters.

8. The system according to claim 1, wherein the output device comprises a second interface coupled to an ablation control device, the second interface is configured to output the ablation parameters to the ablation control device for use during an ablation process.

9. The system according to claim 1, wherein the calculation device calculates the ablation parameters of the ablation probe according to the following quantitative relationship between the ablation range and the ablation parameters of the ablation probe:
Xₚ=Yₚ=c₁-c₂×c₃^{t}
Zₚ=c₄-c₅×c₆^{t}
wherein, c₁, c₂, c₃, c₄, c₅, c₆, c₇ are fixation factors related to the type of the ablation probe.

10. The system according to claim 1, wherein the ablation comprises radiofrequency ablation, cryoablation, microwave ablation, and multi-modal ablation.

11. An intraoperative fast planning method for ablation therapy, comprising:
obtaining a boundary of the tumor and an actual position of an ablation probe relative to a tumor;
obtaining a quantitative relationship between the ablation range and the ablation parameters of the ablation probe;
obtaining maximum ablation range parameters of the ablation probe;
calculating a target treatment area according to the boundary of the tumor and the actual position of the ablation probe relative to the tumor, and determining whether the maximum ablation range of the ablation probe can cover the target treatment area in combination with the maximum ablation range parameters; and
if the maximum ablation range can cover the target treatment area, calculating ablation parameters for the ablation probe according to the actual position of the ablation probe and the target treatment area as well as the quantitative relationship between the ablation range and the ablation parameters of the ablation probe, and outputting the ablation parameters.

12. The method according to claim 11, further comprising: outputting information indicating a need to supplement probe or a change in the position of the probe via the output device if the maximum ablation range cannot cover the target treatment area.

13. The method according to claim 11, further comprising: calculating a supplementary probe position or a changed probe position according to the target treatment area if the maximum ablation range cannot cover the target treatment area, and outputting the supplementary probe position or the changed probe position via the output device.

14. An intraoperative fast planning system for ablation therapy, comprising:
an input device, configured to obtain a boundary of the tumor and an actual position of an ablation probe relative to a tumor, a quantitative relationship between an ablation range and ablation parameters of the ablation probe, and maximum ablation range parameters of the ablation probe.
an output device, configured to output the ablation parameters;
a memory, configured to store computer-executable instructions; and
a processor, configured to implement the steps in the method according to claim 11, when executing the computer-executable instructions.

15. A computer-readable medium, the computer-readable storage medium stores computer-executable commands which are executed by a processor to implement the steps in the method according to claim 11.
